SEQUENCE LISTING (9 sequences)

SEQ ID NO: 1 - DNA, 71 bp, Artificial Sequence
  Synthetic oligonucleotide (pGEX 4T-1-F)
ggttccgcgtggatccgaatatatttttccacagcaatttgatgcggccgctcgagttacagcactttcgt

SEQ ID NO: 2 - DNA, 9 bp, Artificial Sequence
  Synthetic oligonucleotide (pGEX 4T-1-R)
ggatgcgat

SEQ ID NO: 3 - DNA, 37 bp, Artificial Sequence
  Synthetic oligonucleotide (pCES208-F)
gactagagtcggatccatggaatatatttttccacag

SEQ ID NO: 4 - DNA, 37 bp, Artificial Sequence
  Synthetic oligonucleotide (pCES208-R)
ccgcggtggcggccgcttacagcactttcgtggatgc

SEQ ID NO: 5 - DNA, 43 bp, Artificial Sequence
  Synthetic oligonucleotide (pYES2.1-F)
tattaagctcgcccttatggaatatatttttccacagcaattt

SEQ ID NO: 6 - DNA, 37 bp, Artificial Sequence
  Synthetic oligonucleotide (pYES2.1-R)
ctcgaagctcgcccttttacagcactttcgtggatgc

SEQ ID NO: 7 - DNA, 37 bp, Artificial Sequence
  Synthetic oligonucleotide (pNZ8148-F)
gcaggcatgcggtaccatggaatatatttttccacag

SEQ ID NO: 8 - DNA, 37 bp, Artificial Sequence
  Synthetic oligonucleotide (pNZ8148-R)
gcttgagctctctagattacagcactttcgtggatgc

SEQ ID NO: 9 - protein, 419 aa, Paenibacillus
MEYIFPQQFMFGSATAAYQVEGNNTNSDFWAEEHAEGSPYQDKSGDAVDHYRLYREDIALMASLGLKAYRFSIEWARIEPAPGQFSLSAIAHYRDVLQACYEHRLTPVVAMHHFSSPQWLMRFGGWGSEEVPERFAKYCEFVFQELGDLIPYVLTFNEVNLPVMLREVFSSIGIIPPVGIDGAAWTAPGWRASAAQLCGTTADRYVTFHMISDEPKIALLMEAHRRARQTIKRLQPNAKVGLSMALSDIQSVPGGEAWAQQKWQQYFEQYLPALEGDDFFGLQNYTREVYGPEGRVTPAADAELTQMKYEYYPEALEQVIRKVAKALTLPIFVTEHGVATDDDERRIEFIRRGLQGVHACLEEGIDVRGYLHWTTFDNFEWNAGYSMRFGLIGVDRTTQERTVKESARYLGRIASTKVL

(19) United States
(12) Patent Application Publication
(10) Pub. No.: US 2019/0112629 A1
(43) Pub. Date: Apr. 18, 2019

(54) METHOD FOR MASS PRODUCTION OF GINSENOSIDE RH2-MIX

(71) Applicant: INTELLIGENT SYNTHETIC BIOLOGY CENTER, Daejeon (KR)

(72) Inventors: Sun-Chang KIM, Daejeon (KR); Wan Taek IM, Gyeonggi-do (KR); Chang-Hao CUI, Daejeon (KR)

(21) Appl. No.: 15/904,088

(22) Filed: Feb. 23, 2018

(30) Foreign Application Priority Data
Oct. 13, 2017 (KR) 10-2017-0133508

(51) Int. Cl. C12P 33/20; A61K 31/704

(52) U.S. Cl. CPC C12P 33/20; A61K 31/704; C12Y 302/01021

(57) ABSTRACT

The present invention relates to a method for mass production of ginsenoside Rh₂-Mix. The present invention includes treating PPD-Mix with an organic acid and heat to obtain Rg₃-Mix and treating the obtained Rg₃-Mix using a recombinant GRAS strain in the Rg₃-Mix to produce Rh₂-Mix, and thereby facilitates the mass production of ginsenoside Rh₂-Mix using β-glucosidase, which has been known to be difficult. Further, the present invention is advantageous in that the Rh₂-Mix can be produced in high yield even at high temperatures, and mass production thereof for industrial purposes is practical as the production process is simple and more economical than direct use of an enzyme.

### TECHNICAL FIELD

The present invention relates to a method for mass production of ginsenoside Rh₂-Mix, comprising 1) treating PPD-Mix with an organic acid to obtain Rg₃-Mix, and 2) treating the Rg₃-Mix obtained in 1) with β-glucosidase; ginsenoside Rh₂-Mix prepared according to the method; and a composition for converting the Rh₂-Mix, comprising the Rg₃-Mix and β-glucosidase.

### BACKGROUND ART

Saponins are substances consisting of diverse ring compounds formed by the non-sugar portion of glycosides which are widely distributed in the plant kingdom. Among saponins, triterpene saponins, which are contained as a major active ingredient in various kinds of ginseng, in particular, as a major physiologically active ingredient in ginseng or red ginseng, have a different chemical structure from saponins found in other plants. In order to distinguish them from other vegetable saponins, such ginseng saponins are called ginsenosides, meaning ginseng glycosides.

Ginsenosides are broadly classified into major and minor ginsenosides; specifically, the major ginsenosides include Rg₁, Re, Rb₁, Rb₂, Rc, Rd, etc., and the minor ginsenosides present in trace amounts in plants include F₂, Rg₃, Rk₁, Rg₅, Rh₁, Rh₂, Rk₂, Rh₃, gypenoside (Gyp) XVII, Gyp LXXV, compounds K, C—K, Mc, and Mc1, etc. The major ginsenosides are known to account for at least 90% of the ginsenosides contained in dried ginseng.

However, due to its high molecular weight of about 1,000 Da, the major ginsenosides have low absorption by the human body and must be converted into minor ginsenosides having higher absorption and pharmaceutical efficacy in order to increase their efficacy. In other words, the major ginsenosides require a conversion process of deglycosylating sugars such as glucose, arabinose, rhamnose, xylose, etc. to effectively exhibit physiological activities in vivo.

Among said various minor ginsenosides, Rh₂(S), Rh₂(R), Rk₂, and Rh₃ are known to be present in ginseng in an infinitesimal amount, and trace amounts thereof can be obtained during the preparation of red ginseng. Nevertheless, the minor ginsenosides have various activities such as protection of brain cells (KR Patent Nos. 10-0759772 and 10-0688425), an anti-cancer effect (Protein & Cell 5.3 (2014): 224-234.), an effect of inhibiting chronic dermatitis (Archives of Pharmacal Research 29.8 (2006): 685-690.), an anti-inflammatory effect (Neurochemical Research 41.5 (2016): 951-957.), etc., and thus are receiving much attention regarding their pharmacological actions. In this regard, there is a need to develop technology for mass production of the ginsenosides.

Recently, a method for chemical decomposition and glycoside synthesis, an enzymatic method, etc. have been suggested as methods for mass production of the minor ginsenosides, which are accompanied by an issue of being uneconomical, as their final yields are low and the production processes are complicated. In particular, there have been many studies on the methods for converting major ginsenosides into minor ginsenosides using enzymes such as β-glucosidase, α-L-arabinopyranosidase, α-L-arabinofuranosidase, α-L-rhamnosidase, etc., but none of these had high efficiency of mass production.

In order to solve such problem, Korean Patent No. 10-2017-0027367 discloses a method for obtaining a minor ginsenoside by obtaining an intermediate using viscozyme instead of the enzyme, followed by treating the ginsenoside F2 with an organic acid; however, the method has a problem of being uneconomical as the enzymes should be consistently purchased in the amount needed for a reaction. Additionally, in the case of using the above-disclosed enzymes, there would be problems in that the level of intermediate metabolites rapidly increases if it deviates from an optimum condition of the enzymes; in particular, yield remarkably decreases when the organic acid is treated at high temperature rather than low temperature. Accordingly, there is still an urgent need for studies on methods for preparing a high yield of minor ginsenosides such as Rh₂ both economically and in such a short period of time.

Under such circumstances, the present inventors endeavored to develop a method for mass production of Rh₂, Rk₂, and Rh₃ present in trace amounts in ginseng, and as a result, ensured that the minor ginsenosides Rh₂, Rk₂, and Rh₃ could be prepared in a massive amount by adding an organic acid to PPD-Mix and treating the obtained Rg₃-Mix with β-glucosidase obtained from a GRAS strain encoding the same, thereby completing the present invention.

### DISCLOSURE

### Technical Problem

An object of the present invention is to provide a method for mass production of ginsenoside Rh₂-Mix, comprising 1) treating PPD-Mix with an organic acid to obtain Rg₃-Mix; and 2) treating the Rg₃-Mix obtained in 1) with β-glucosidase.

Another object of the present invention is to provide Rh₂-Mix prepared according to the method.

Still another object of the present invention is to provide a composition for converting the Rh₂-Mix, comprising the Rg₃-Mix and β-glucosidase.

### Technical Solution

As an aspect, the present invention provides a method for mass production of ginsenoside Rh₂-Mix, comprising 1) treating PPD-Mix with an organic acid to obtain Rg₃-Mix; and 2) treating the Rg₃-Mix obtained in 1) with β-glucosidase.

Hereinbelow, the method for mass production of ginsenoside Rh₂-Mix will be described in detail.

As used herein, the term “ginsenoside” refers to a saponin in ginseng. The ginseng saponin is a triterpene saponin which has a unique chemical structure different from saponins found in other plants, and due to its unique pharmaceutical efficacy, it is called ginsenoside, meaning ginseng glycoside.

The ginsenosides can be classified into three types according to the constitution of aglycone: protopanaxadiol-type (PPD-type) ginsenosides, protopanaxatriol-type (PPT-type) ginsenosides, and oleanolic acid-type ginsenosides. To date, more than 180 ginsenosides have been identified from ginseng, most of which are PPD-type ginsenosides.

The three groups of ginsenosides are classified according to the position and number of sugar moieties attached by glycosidic bonds at C3, C6, and C20 of a ring within the chemical structure.

Specifically, the PPD-type ginsenoside, as a dammarane-type saponin, refers to PPD having —OH groups at positions of C3, C12, and C20 or a ginsenoside glycosylated at one or more —OH groups of the PPD. The backbone thereof is as shown in Formula 1. Specific examples include Rb₁, Rb₂, Rb₃, Rc, Rd, gypenoside XVII, compound O, compound Mc1, F₂, compound Y, compound Mc, Rg₃, Rh₂, C—K, etc.

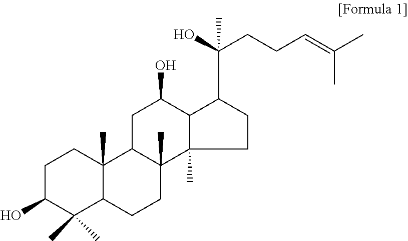

Additionally, the PPT-type ginsenoside, as a dammarane-type saponin, refers to PPT having —OH groups at positions of C3, C6, C12, and C20 or a ginsenoside glycosylated at the —OH group of the PPT. The backbone thereof is as shown in Formula 2. Examples of the PPT-type ginsenoside include Re, Rg₁, Rf, Rg₂, PPT, Rh₁, etc.

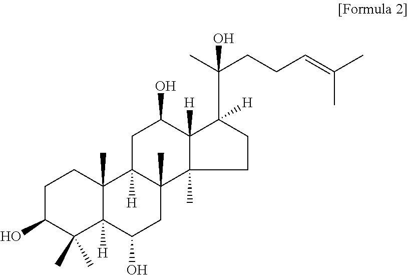

Additionally, the oleanolic acid-type ginsenoside has a pentacyclic backbone as shown in Formula 3 below, and ginsenoside Ro is the only example.

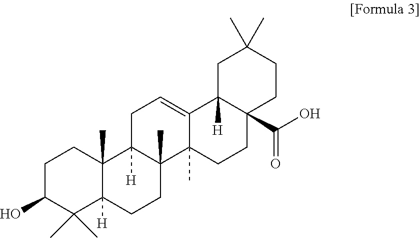

As used herein, the term “Rh₂-Mix”, as an Rh₂-type minor ginsenoside, refers to a minor ginsenoside among those in the form in which a single glucose moiety is attached to the C3 position of the basic carbon backbone of the PPD-type ginsenoside. Specifically, the Rh₂-Mix may be Rh₂, Rk₂, or Rh₃, more specifically, may be 20(S)—Rh₂, 20(R)—Rh₂, Rk₂, or Rh₃, but is not limited thereto.

The minor ginsenoside, which accounts for a low percentage of the ginsenoside of ginseng, is a minor ginsenoside produced by hydrolyzing glucose, xylose, Glc(1→6)Glc, or Xly(1→6)Glc positioned at C20 of a PPD- or PPT-type ginsenoside which is a major ginsenoside having low absorption and being readily absorbed by the human body. In the present invention, the minor ginsenoside refers to a minor ginsenoside in which a single glucose moiety is attached to the C3 position of the basic carbon backbone of the PPD-type ginsenoside.

The term “Glc(1→6)Glc” refers to a disaccharide in which C1 on a glucose moiety is linked to C6 on another glucose moiety via an α or β linkage, and the term “Xyl(1→6)Glc” refers to a disaccharide in which C1 on a xylose molecule is linked to C6 on a glucose moiety via an α or β linkage.

The ginsenoside “Rh₂”, as shown in Formula 4 below, is a minor ginsenoside in which one glucose molecule is attached to the C3 position of the basic carbon backbone of the PPD-type ginsenoside, and compared to Rb₁, Rd, Rb₃, etc., it is more easily absorbed by the human body due to the removal of the sugars. The ginsenoside Rh₂ includes Rh₂(S) and Rh₂(R).

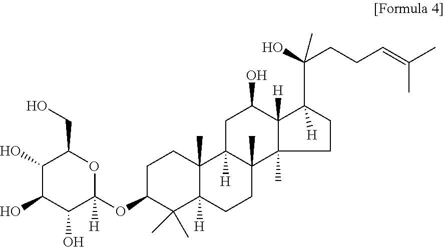

The ginsenoside “Rk₂”, as shown in Formula 5 below, is a minor ginsenoside in which one glucose molecule is attached to C3 of the basic carbon backbone of the PPD-type ginsenoside and there is a double bond between C20 and C21. Compared to Rk₁, Rb₁, Rd, Rb₃, etc., it is more easily absorbed by the human body due to the removal of the sugars.

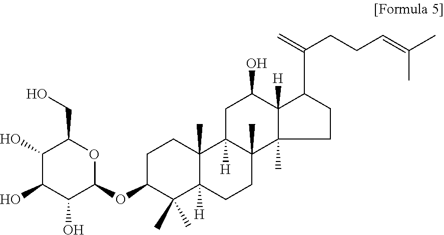

The ginsenoside “Rh₃”, as shown in Formula 6 below, is a minor ginsenoside in which one glucose molecule is attached to C3 of the basic carbon backbone of the PPD-type ginsenoside and there is a double bond between C20 and C22. Compared to Rh₁, Rb₁, Rd, Rb₃, etc., it is more easily absorbed by the human body due to the removal of the sugars.

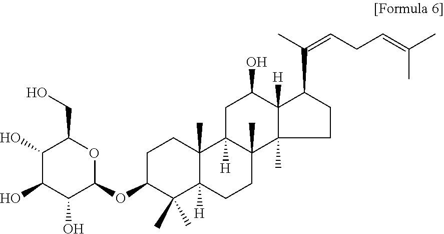

The method for mass production of Rh₂-Mix of the present invention includes treating PPD-Mix with an organic acid to obtain Rg₃-Mix as step 1).

As used herein, the term “PPD-Mix”, as a PPD-type ginsenoside, refers to a major ginsenoside PPD among the PPD-type ginsenosides, in which at least two excess glucose molecules are attached to the basic carbon backbone of the PPD-type ginsenoside. Specifically, the PPD-Mix is not limited, but may be Rb₁, Rb₂, Rb₃, Rc, Rd, and F₂, and more specifically Rb₁, Rb₂, Rb₃, Rc, and Rd.

The major ginsenosides refer to ginsenosides that account for a high percentage of ginseng ginsenosides, and due to the high molecular weight compared to the minor ginsenosides, have low in vivo absorption.

As used herein, the term “organic acid” is a general term for organic compounds which show acidic properties, and refers to a substance containing a carboxyl group or sulfonic group, excluding some acids such as uric acid. Specifically, the organic acids are not necessarily limited thereto, but may include acetic acid, oxalic acid, tartaric acid, benzoic acid, butyric acid, palmitic acid, ascorbic acid, citric acid or uric acid, and more specifically, citric acid.

The organic acids are added in a concentration of 0.5% to 5%, specifically 1% to 3%, more specifically 1.5% to 2.5%, but are not limited thereto.

Step 1) may further comprise heating. Specifically, the heating may be performed before or after the organic acid treatment, more specifically, the treatment of an organic acid in the PPD-Mix.

As used herein, the term “heating” refers to applying heat to a substance, and can be interchangeably used with “heat treatment”.

A temperature of the heating may be 100° C. to 140° C., specifically 110° C. to 130° C., more specifically 115° C. to 125° C., but is not limited thereto. In particular, the present invention shows in a specific exemplary embodiment that the Rh₂-Mix could be obtained with a yield efficiency of about 50% despite the treatment with an organic acid followed by heat treatment at a high temperature of 100° C. or above (FIG. 5). This result resolved a problem of the Rh₂-Mix yield efficiency significantly lowered upon treating at a high temperature, indicating that high yield of the Rh₂-Mix can be obtained at not only a low temperature but also a high temperature.

Additionally, the heat treatment time may be in the range of 1 minute to 60 minutes, specifically 5 minutes to 30 minutes, more specifically 10 minutes to 20 minutes, but is not limited thereto.

In a specific exemplary embodiment of the present invention, for the mass production of Rh₂-Mix, the organic acid and heat treatments were conducted at 121° C. for 15 minutes followed by treating with β-glucosidase to prepare Rh₂-Mix. As a result, the yield of the final product Rh₂-Mix was measured to be about 50% despite the two treatments at a high temperature, indicating that compared to previous studies which had lowered yield due to intermediates produced during rapid treatment at a high temperature, high yield can be achieved even at a high temperature (FIG. 5).

As used herein, the term “Rg₃-Mix”, as an Rg₃-type minor ginsenoside, refers to a minor ginsenoside among those in the form in which two glucose moieties (Glc→Glc) are attached to the C3 position of the basic carbon backbone of the PPD-type ginsenoside. Specifically, the Rg₃-Mix may be Rg₃, Rk₁, or Rg₅, more specifically, 20(S)-Rg₃, 20(R)-Rg₃, Rk₁, or Rg₅.

As used herein, ginsenoside “Rg₃”, as shown in Formula 7 below, is a minor ginsenoside in the form in which two glucose moieties (Glc→Glc) are attached to the C3 position of the basic carbon backbone of the PPD-type ginsenoside. Compared to Rb₁, Rb₂, Rb₃, Rc, Rd, and other major ginsenosides, it is more easily absorbed by the human body due to the removal of the sugars. The ginsenoside Rg₃ may include Rg₃(S) and Rg₃(R).

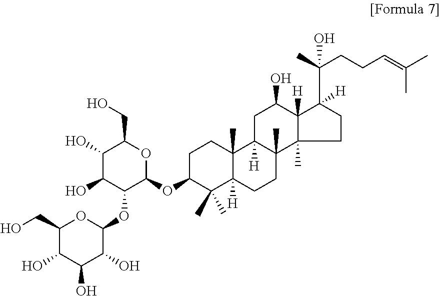

The Rg₃-Mix of the present invention is obtained by treating the PPD-Mix with an organic acid, and may be an enzymatic reaction substrate for β-glucosidase that is treated after step 1).

As used herein, the term ginsenoside “Rk₁”, as shown in Formula 8 below, is a minor ginsenoside in which two glucose moieties (Glc→Glc) are attached to the C3 position of the basic carbon backbone of the PPD-type ginsenoside and where there is a double bond between C20 and C21. Compared to Rb₁, Rd, Rb₃, and other major ginsenosides, it is more easily absorbed by the human body due to the removal of the sugars.

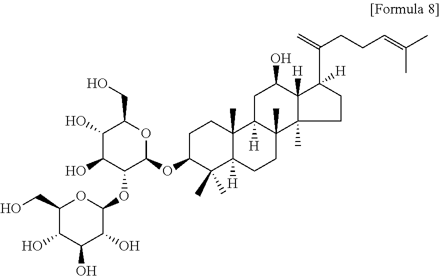

As used herein, the term ginsenoside “Rg₅”, as shown in Formula 9 below, is a minor ginsenoside in which two glucose moieties (Glc→Glc) are attached to the C3 position of the basic carbon backbone of the PPD-type ginsenoside and where there is a double bond between C20 and C22. Compared to Rb₁, Rd, Rb₃, and other major ginsenosides, it is more easily absorbed by the human body due to the removal of the sugars.

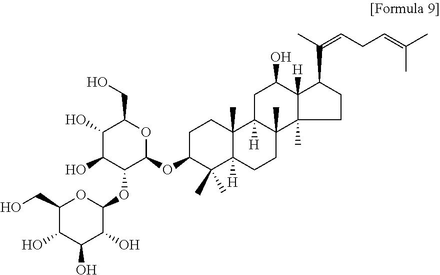

In a specific exemplary embodiment of the present invention, citric acid was treated in the PPD-Mix which includes Rb₁, Rc, Rd, and Rb₂ to remove the glucose attached to C20, and Rg₃-Mixes of 20(S)-Rg₃, 20(R)-Rg₃, Rk₁, and Rg₅ were obtained.

Consequently, the process of obtaining Rg₃-Mix from PPD-Mix through the organic acid treatment could be identified.

The method for mass production of Rh₂-Mix of the present invention includes treating the Rg₃-Mix obtained in 1) with β-glucosidase as step 2).

As used herein, the term “Rg₃-Mix” is the same as previously described.

As used herein, the term “β-glucosidase” is a general term for enzymes which has hydrolytic activity showing absolute specificity to β-D-glucopyranoside, specifically, enzymes which hydrolyze a glycosidic bond of ginsenoside by specifically reacting with ginsenosides. Specifically, the β-glucosidase may be a protein consisting of the amino acid sequence of SEQ ID NO: 9, and includes an amino acid having a homology to the above sequence of about 80% or above, specifically 90% or above, more specifically 95% or above, and most specifically 99% or above without limitation. Additionally, any amino acid sequence can be included in the scope of the present invention as long as it exhibit β-glucosidase activity.

In the present invention, the Rg₃-Mix is converted to the Rh₂-Mix by hydrolyzing one glucose moiety at C20 of the Rg₃-Mix. Specifically, 20(S)-Rg₃, 20(R)-Rg₃, Rk₁, and Rg₅ can be converted to 20(S)—Rh₂, 20(R)—Rh₂, Rk₂, and Rh₃ respectively, but are not limited thereto.

The β-glucosidase may be obtained from a recombinant GRAS (Generally Recognized As Safe) strain.

The “recombinant GRAS strain” refers to a strain in which genetic traits are recombined by introducing a desired gene, and may be one selected from the group consisting of Corynebacterium sp., Saccharomyces sp., and Lactococcus sp., but is not limited thereto.

The Corynebacterium sp. may be C. amycolatum, C. aquaticum, C. bovis, C. diphtheria, C. glutamicum, C. granulosum, C. minutissimum, C. parvum, C. pseudotuberculosis, C. renale, C. ulcerans, and C. urealyticum, and specifically, may be Corynebacterium glutamicum.

The Saccharomyces sp. may be S. arboricolus, S. bayanus, S. boulardii, S. bulderi, S. cariocanus, S. cariocus, S. cerevisiae, S. chevalieri, S. dairenensis, S. elhpsoideus, S. eubayanus, S. exiguous, S. florentinus, S. fragilis, S. kluyveri, S. kudriavzevii, S. martiniae, S. mikatae, S. monacensis, S. norbensis, S. paradoxus, S. pastorianus, S. spencerorum, S. turicensis, S. unisporus, S. uvarum, and S. zonatus, and specifically, may be Saccharomyces cerevisiae.

The Lactococcus sp. may be L. chungangensis, L. formosensis, L. fujiensis, L. garvieae, L. lactis, L. piscium, L. plantarum, L. raffinolactis, and L. taiwanensis, and specifically, may be Lactococcus lactis.

The GRAS strain may be a transformant into which a vector which includes a nucleic acid encoding β-glucosidase protein is introduced.

The “transformant” refers to an organism into which a foreign gene is introduced. The term “transformation” refers to genetic modification of a trait of an organism or cell by exogenous DNA.

In a specific exemplary embodiment of the present invention, a ginsenoside-transforming β-glucosidase gene obtained from genomic DNA of Paenibacillus mucilaginosus is introduced into a vector, and the vector was introduced into the three types of the GRAS strains, Corynebacterium sp., Saccharomyces sp., and Lactococcus sp., to prepare a transformant.

In step 2) of the present invention, β-glucosidase can be treated at a pH range of 5.5 to 7.5, specifically 5.8 to 7.2, more specifically 6.0 to 7.0, but is not limited thereto.

Additionally, the treatment time may be 12 hours to 36 hours, specifically 18 hours to 30 hours, more specifically 20 hours to 28 hours, but is not limited thereto.

In a specific exemplary embodiment of the present invention, β-glucosidase was treated in the Rg₃-Mix and reacted at pH 6.5 for 24 hours, thereby confirming that the Rg₃-Mix was completely converted to Rh₂-Mix. In particular, when β-glucosidase was treated, it was confirmed that the mass production of Rh₂-Mix was feasible with a yield of about 50%.

Such result indicates that the Rh₂-Mix could be prepared in a massive amount with high yield using the preparation method provided in the present invention.

As still another aspect, the present invention provides Rh₂-Mix prepared by the above-described method.

The term “Rh₂-Mix” is the same as previously described.

Specifically, the Rh₂-Mix, examples of which are 20(S)—Rh₂, 20(R)—Rh₂, Rk₂, and Rh₃, due to its small molecular weight compared to existing major ginsenosides, has high absorbance, leading to high pharmaceutical activity.

As still another aspect, the present invention provides a composition for converting ginsenoside Rh₂-Mix, comprising Rg₃-Mix and β-glucosidase.

The terms “Rg₃-Mix”, “PPD-Mix”, “Rh₂-Mix”, and “β-glucosidase” are the same as previously described.

Due to its specific activity to ginsenoside, the β-glucosidase is useful in the conversion of Rg₃-Mix consisting of 20(S)-Rg₃, 20(R)-Rg₃, Rk₁, and Rg₅ to Rh₂-Mix.

In a specific exemplary embodiment of the present invention, β-glucosidase was shown to have excellent conversion activity by confirming that Rg₃-Mix consisting of 20(S)-Rg₃, 20(R)-Rg₃, Rk₁, and Rg₅, after treatment with β-glucosidase, was completely converted to Rh₂ (FIG. 3).

As still another aspect, the present invention provides an anti-cancer pharmaceutical composition comprising the Rh₂-Mix prepared by the preparation method of the present invention.

The “Rh₂” is the same as previously described.

As used herein, the term “anti-cancer” refers to all behaviors of suppressing or delaying the onset of a cancer by administering the composition to a subject, or of improving or alleviating symptoms of the cancer by administering the composition to a subject suspected of developing the cancer.

The pharmaceutical composition of the present invention may be used as a single formulation or prepared as a combination formulation further including a drug known to have an approved anti-cancer effect. Using a pharmaceutically acceptable carrier or excipient, the pharmaceutical composition may be formulated in a unit dosage form or prepared by encapsulating the same in a multiple dose container.

As used herein, the term “pharmaceutically acceptable carrier” may refer to a carrier or a diluent that does not inhibit biological activities and properties of a compound to be administered without irritating an organism. A type of the carrier which can be used in the present invention is not particularly limited, and any carrier can be used as long as it is a pharmaceutically acceptable carrier commonly used in the art. Non-limiting examples of the carrier include saline, sterile water, Ringer's solution, buffered saline, albumin injection solution, dextrose solution, maltodextrin solution, glycerol, ethanol, etc. These can be used alone or in a combination of two or more. The carrier may include a non-naturally occurring carrier.

Additionally, other conventional additives such as antioxidants, buffers, and/or bacteriostats may be further included, if necessary. By further adding diluents, dispersants, surfactants, binders, lubricants, etc., the pharmaceutical composition may be formulated into an injectable formulation (e.g., aqueous solution, suspension, and emulsion), pills, capsules, granules, or tablets.

The pharmaceutical composition of the present invention may include an effective amount of Rh₂-Mix. As used herein, the term “pharmaceutically effective amount” refers to an amount sufficient to treat a disease at a reasonable benefit/risk ratio that is applicable to any medical treatment, and can generally be administered in an amount of 0.001 mg/kg to 1000 mg/kg, preferably 0.05 mg/kg to 200 mg/kg, more preferably 0.1 mg/kg to 100 mg/kg once daily or several times in divided doses. With respect to the purpose of the present invention, however, it is preferable that a specific pharmaceutically effective amount for a specific patient vary depending on the type and degree of a reaction to be achieved in the treatment, specific composition (whether another agent is included in the composition), the patient's age, body weight, general health conditions, gender, and diet, administration time, administration route, the composition's secretion ratio and treatment period, and other drugs used together or simultaneously with the specific composition, as well as a variety of factors, and analogous factors well known in the medical field.

The pharmaceutical composition of the present invention can be administered as an independent therapeutic drug or co-administered with other therapeutic drugs; sequentially or simultaneously administered with existing therapeutic drugs; and once or several times. Considering all of the above factors, it is important to dose the minimum amount that can achieve the maximum effect with no side effects, which can be readily determined by one of ordinary skill in the art.

As used herein, the term “administration” refers to introduction of the pharmaceutical composition of the present invention to a patient by an appropriate manner, and the composition may be administered via various oral or parenteral routes as long as it can arrive at a target tissue.

The method for administering the pharmaceutical composition according to the present invention is not particularly limited and can be any method conventionally used in the related art. Unlimited examples of the administration method include oral and parenteral administrations. The pharmaceutical composition according to the present invention can be prepared in various formulations according to a target administration method.

The administration frequency of the composition of the present invention is not particularly limited, but may be once daily or several times in divided doses.

As used herein, the term “pharmaceutically acceptable salt” refers to a formulation that does not abrogate biological activity and properties of the administered Rh₂-Mix. The pharmaceutically acceptable salts include acids forming non-toxic acid addition salts containing a pharmaceutically acceptable anion; for example, acid addition salts formed by inorganic acids such as hydrochloric acid, sulfuric acid, nitric acid, phosphoric acid, hydrobromic acid, hydroiodic acid, etc., organocarbonic acids such as tartaric acid, formic acid, citric acid, acetic acid, trichloroacetic acid, trifluoroacetic acid, gluconic acid, benzoic acid, lactic acid, fumaric acid, maleic acid, salicylic acid, etc., and sulfonic acid such as methanesulfonic acid, ethanesulfonic acid, benzenesulfonic acid, p-toluenesulfonic acid, etc. For example, pharmaceutically acceptable carboxylic acid salts include alkaline earth metal salts or metal salts formed by lithium, sodium, potassium, calcium, magnesium, etc., amino acid salts such as lysine, arginine, guanidine, etc., and organic salts such as dicyclohexylamine, N-methyl-D-glucamine, tris(hydroxymethyl)methylamine, diethanolamine, choline, triethylamine, etc.

A specific exemplary embodiment confirmed that a massive amount of the Rh₂-Mix was obtained by treating the Rg₃-Mix consisting of 20(S)-Rg₃, 20(R)-Rg₃, Rk₁, and Rg₅ with β-glucosidase. Accordingly, the Rh₂-Mix prepared according to the method of the present invention can be suggested as an anti-cancer pharmaceutical composition.

As still another aspect, the present invention provides an anti-inflammatory composition comprising the Rh₂-Mix.

The term “Rh₂-Mix” is the same as previously described.

As used herein, the term “anti-inflammation” may refer to all behaviors of suppressing or delaying the onset of inflammation by administering the composition to a subject, or of improving or alleviating symptoms of the inflammation by administering the composition to a subject suspected of developing the inflammation.

A specific exemplary embodiment confirms that a massive amount of the Rh₂-Mix was obtained by treating the Rg₃-Mix consisting of 20(S)-Rg₃, 20(R)-Rg₃, Rk₁, and Rg₅ with β-glucosidase. Accordingly, the Rh₂-Mix prepared according to the method of the present invention can be suggested as an anti-inflammatory pharmaceutical composition.

Such result indicates that the stable and economical mass production of the minor ginsenosides is feasible through the method for mass production of ginsenoside Rh₂-Mix provided in the present invention, and that the thus-prepared Rh₂-Mix is available for various uses, e.g., as a pharmaceutical composition, anti-inflammatory composition, etc.

### Advantageous Effects

In the present invention, the Rh₂-Mix is prepared by conducting the organic acid and heat treatments in the PPD-Mix to obtain the Rg₃-Mix and treating the β-glucosidase obtained using the recombinant GRAS strain in the Rg₃-Mix, thereby facilitating mass production of Rh₂-Mix using β-glucosidase, which was previously known to be difficult. Additionally, the present invention allows the high yield of Rh₂-Mix production, and has an advantage in that mass production thereof for industrial purposes is practical as the production process is simple and more economical than direct use of an enzyme.

### DESCRIPTION OF THE DRAWINGS

FIGS. 1A-B shows the result of SDS-PAGE analysis for confirmation of protein expression of the recombinant E. coli and GRAS host strains; A: lane 1, molecular weight standard; lane 2, soluble crude extract of recombinant E. coli without induction; lane 3, BglPm of recombinant E. coli after induction; lane 4, purified soluble fraction of recombinant E. coli (BglPm); lane 5, non-inducible fraction of Corynebacterium glutamicum harboring pCES208; lane 6, inducible BglPm_C; lane 7, purified BglPm_C (C. glutamicum); lane 8, molecular weight standard. B: lane 9, molecular weight standard; lane 10, non-inducible fraction of Saccharomyces cerevisiae; lane, 11 inducible BglPm_S; lane 12, BglPm_S protein of S. cerevisiae after purification; lane, 13-14, non-inducible and inducible fraction of Lactococcus lactis; lane 15, molecular weight standard.

FIGS. 2A-D are graphs showing the effect of sonication on the enzyme activity of each recombinant enzyme of: 2a, E. coli; 2b, C. glutamicum; 2c, S. cerevisiae; 2d, Lactococcus lactis.

FIGS. 3A-B are results showing TCL analysis of a time course of ginsenosides by acid and enzyme treatments; A, transformation of ginsenoside PPD-Mix; B, transformation of Rg₃-Mix.

FIGS. 4A-D are results showing HPLC analysis of the transformation of the ginsenosides PPD-Mix and Rg₃-Mix by acid and enzyme treatments; A, ginsenosides standard; B, PPD-Mix as a substrate; C, converted Rg₃-Mix 15 min after acid treatment in PPD-Mix at 121° C.; D, converted Rh₂-Mix after 24 h of the reaction of BglPm_C with Rg₃-Mix.

FIG. 5 is a schematic view of transformation pathways for Rh₂-Mix production and the relative structures of ginsenosides.

FIG. 6 is a schematic diagram showing the entire process of Rh₂-Mix production from PPD-Mix as a substrate using the combined method of acid treatment and enzyme treatment.

### MODE FOR INVENTION

Hereinbelow, the present invention will be described in detail with accompanying exemplary embodiments. However, the exemplary embodiments disclosed herein are only for illustrative purposes and should not be construed as limiting the scope of the present invention.

### Example 1. Preparation of Recombinant Expression Vector and Transformed Microorganism

Ginsenosides standards, Rb₁, Rc, Rb₂, Rd, 20(S)-Rg₃, 20(R)-Rg₃, 20(S)—Rh₂, F₂, and CK, were bought from Nanjing Zelang Medical Technology Co., Ltd. (China), while ginsenosides 20(R)—Rh₂, Rk₁, Rg₅, Rk₂, and Rh₃ were purchased from Chengdu Biopurify Phytochemicals Co., Ltd. (China).

The PPD-Mix-type ginsenosides mixture from the root of American root saponins Panax quinquefolius, containing Rb₁ (328 mg/g), Rc (173 mg/g), Rd (107 mg/g) and small amounts of Rb₂ (25 mg/g) and Rb₃ (25 mg/g), acquired from Hongjiou Biotech Co. Ltd. (China), was used as the initial substrate. The genomic DNA from Paenibacillus mucilaginosus KCTC 3870^{T}, E. coli, and pGEX 4T-1 plasmid (GE Healthcare, USA) were used for the β-glucosidase gene, host, and expression vector sources, respectively. P. mucilaginosus KCTC 3870^{T} was grown in aerobic conditions at 37° C. on nutrient agar (NA, BD, USA). The recombinant E. coli for protein expression was cultivated in a Luria-Bertani (LB) medium supplemented with ampicillin (100 mg/L). C. glutamicum and the pCES208 plasmid, S. cerevisiae and pYES 2.1 plasmid, L. lactis strain NZ9000 and PNZ8148 plasmid (MoBiTec GmbH, Germany) were used as hosts and expression vector sources, respectively (Table 1).

TABLE 1

| | | Sources or |
|---|---|---|
| | Relevant genotype or description | references |
| Hosts | | |
| BL21 (DE3) | fhuA2 [Ion] ompT gal (λ DE3) [dcm] ΔhsdS λ DE3 = λ | NEB strain catalog |
| | sBamHlo ΔEcoRl-B int::(lacl::PlacUV5::T7 gene1) i21 Δnin | no. C2527 |
| BL21(DE3) harboring | Cloned with glucoside hydrolyzed (BglPm) gene for | |
| pGEX-BglPm | ginsenosides transformation | |
| C. glutamicum ATCC | Biotin-auxotrophic wild type | ATCC |
| 13032 | | |
| C. glutamicum WJ001 | ATCC 13032, cloning host for ginsenosides transformation | This study |
| S. cerevisiae CEN. | MATa ura3-52 MAL2-8^{c} SUC2 | |
| PK113-5D | | |
| S. cerevisiae CEN. | Cloning host for ginsenosides transformation | This study |
| PK113-5D | | |
| L. lactis pNZ8148 | Cloning host for ginsenosides transformation | MoBiTec |
| Plasmids | | |
| pGEX-BglPm | Harboring β-glucosidase (BglPm) gene | |
| pCES208 | E. coli/C. glutamicum shuttle vector, 5.93 kb, Kan^{R} | |
| pCES208 | Expression vector for His-tag fusion in C. glutamicum | This study |
| | ATCC 13032 with β-glucosidase (BglPm) gene; Kan^{R} | |
| pYES2 | Ap^{R} URA3 GALp | Invitrogen |
| | | Corporation |
| pYES2.1 | pYES2.1 TOPO TA ® vector | |
| pYES2.1 | Expression vector for BglPm gene in S. cerevisiae 1389 | This study |
| pNZ8148 | Broad-host-range vector; Cm^{R}, PnisA | MoBiTec |
| pNZ8148 | Expression vector for BglPm gene in L. lactis | This study |

CECT, Coleccioèn Espanìola de Cultivos Tipo;
YGSC, Yeast Genetic Stock Center, Berkeley, Ca, USA.
Kan^{R}, kanamycin resistance
Cm^{R}, chloroampinicol resistance

### Example 2. Preparation of Ginsenoside Rg₃-Mix Through Acid Treatment of PPD-Mix

In order to prepare Rg₃-Mix for use in the enzyme reaction, heat treatment with an organic acid was used. The PPD-Mix was dissolved in distilled water at a concentration of 50 g/L and included citric acid (2%, w/v) and heat-treated (121° C. for 15 min) to prepare the ginsenosides Rg₃-Mix [20(S)-Rg₃ (118.6 mg/g), 20(R)-Rg₃ (108.8 mg/g), Rk₁ (144.9 mg/g), and Rg₅ (170.5 mg/g)] from PPD-Mix. After the reaction, the resultant Rg₃-Mix was used as the substrate for the subsequent enzyme reaction.

### Example 3. Preparation of GRAS Strain Having Recombinant BglPm

The genomic DNA from Paenibacillus mucilaginosus KCTC 3870^{T} was extracted using a genomic DNA extraction kit (Solgent, Korea). The gene encoding β-glucosidase, which has ginsenoside-transforming activity, was amplified from the extracted genomic DNA as a template via polymerase chain reaction (PCR) using Pfu DNA polymerase (Solgent, Korea). The sequence of the oligonucleotide primers used for the gene cloning was based on the DNA sequence of BglPm (β-glucosidase; GenBank accession number: AEI42200). Four sets of primers (Table 2) were designed and synthesized to amplify the gene of BglPm for E. coli and three kinds of GRAS strains. The amplified DNA fragment obtained from the PCR was purified and inserted into the pGEX 4T-1 GST fusion vector, pYES2.1 His-tag combined vector, pCES208 His-tag combined vector, and pNZ8148 vector, respectively, using an EzCloning Kit (Enzynomics Co. Ltd., Korea). By introducing the resulting recombinant pGEX-BglPm, pYES2.1-BglPm, pCES208-BglPm, and pNZ8148-BglPm into E. coli BL21 (DE3), C. glutamicum, S. cerevisiae, and L. lactis strains, respectively, E. coli strain BL21 and the three GRAS hosts strains were constructed with different vector systems.

TABLE 2

| Function and primer | Sequence |
|---|---|
| GST-pGEX 4T-1 fusion construction | |
| pGEX-4T-1-F | G GTT CCG CCGT GGA TCC GAA TAT ATT TTT CCA |
| | CAG CAA TTT CATG CGG CCG CTC GAG TTA CAG |
| | CAC TTT CGT |
| pGEX-4T-1-R | GGA TGC GAT |
| His tag-pCES208 and pYES2.1 fusion | |
| construction | |
| pCES208-F | GACTAGAGTCGGATCC ATG GAATATATTTTTCCACAG |
| pCES208-R | CCGCGGTGGCGGCCGC TTA CAGCACTTTCGTGGATGC |
| pYES2.1-F | TATTAAGCTCGCCCTTATGGAATATATTTTTCCACAGCAATTT |
| pYES2.1-R | CTCGAAGCTCGCCCTTTTACAGCACTTTCGTGGATGC |
| β-glucosidase fusion construction | |
| pNZ8148-F | GCAGGCATGCGGTACCATG GAATATATTTTTCCACAG |
| pNZ8148-R | GCTTGAGCTCTCTAGATTA CAGCACTTTCGTGGATGC |

(Top to Bottom: SEQ ID NOS: 1-8)

### Example 4. Expression and Purification of BglPm within the GRAS Strain

To determine the expression level of the GRAS host strains and amount of soluble protein, the induction of expression of recombinant E. coli and the three GRAS hosts was studied. The recombinant E. coli was cultivated in LBA (Luria-Bertani with ampicillin [100 mg/L final concentration]) and induced by 0.15 mM IPTG at 28° C. Similarly, C. glutamicum, S. cerevisiae, and L. lactis were cultivated in LBK (Luria-Bertani with kanamycin [50 mg/L final concentration] induced by glucose [10 g/L final]), YPD (galactose inducible [18 g/L final concentration]), and GM-17 [glucose 10 g/L and induced by nisin, 10 μL/L final concentration)] at 30° C., respectively, and were then induced.

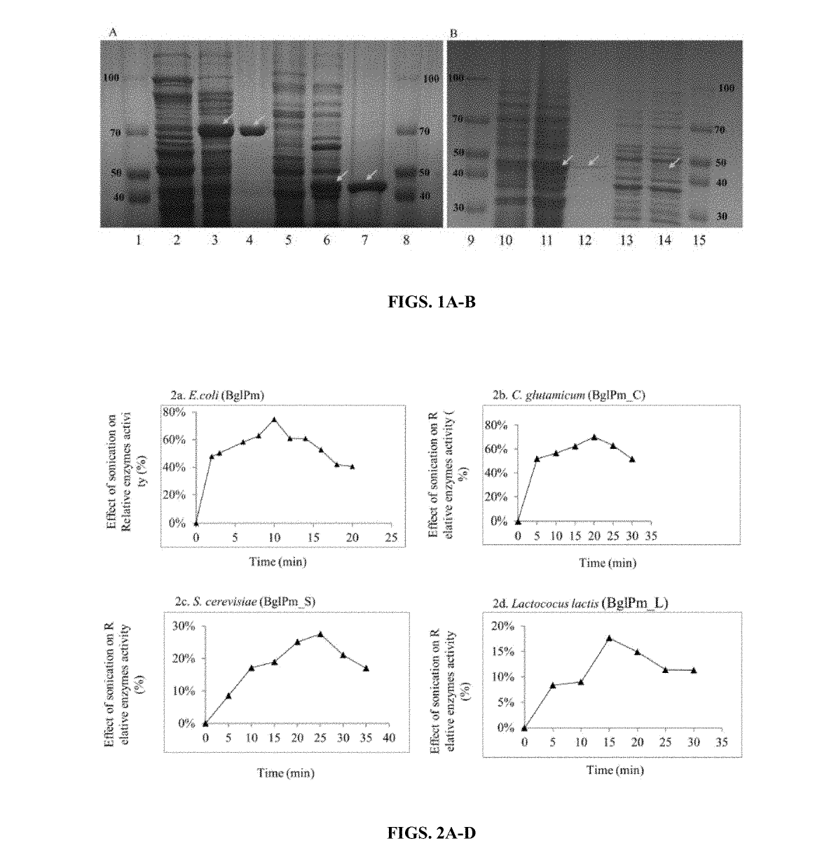

In order to confirm the protein expression of the induced strain, SDS-PAGE analysis was performed using a 10% acrylamide-bis-acrylamide gel (37.5:1 [Qbiogene]). Culture samples were prepared by mixing dye with the samples of each cell suspension at a ratio of 3:1. The solutions were mixed well and heated for 5 min at 100° C. Similarly, 15 μL of dye-sample mixture was loaded in each lane of the gel and electrophoresis was performed in SDS-Tris-Glycine buffer at a constant voltage until the dye front reached the bottom of the gel. The protein bands were stained with Coomassie brilliant Blue Ez stain (AQua), and de-stained in distilled water. After de-staining, the results of the GRAS host strains were compared with those of the recombinant E. coli (FIGS. 1a and 1b).

As a result of the comparison, the molecular masses of the native β-glucosidase, calculated via an amino acid sequence and fusion tag protein expressed in E. coli, C. glutamicum, S. cerevisiae, and L. lactis, were found to be 72 (46+26) kDa, 47 (46+1) kDa, 47 (46+1) kDa, and 46 kDa, respectively (Table 3).

The GST-BglPm and His-tag-BglPm were purified using the GST and His-tag binding resin column (Elpis Biotech). After purification of cell lysates, non-induced, induced, and purified protein soluble fractions were analyzed by SDS-PAGE, and the prominent protein bands, with an apparent molecular weight near 72 kDa, 47 kDa, 47 kDa, and 46 kDa, were identified in the three GRAS host strains and recombinant E. coli lysates. In the comparative study of SDS-PAGE assay of the GRAS host strains with E. coli (FIG. 1a, lanes 3 and 4), it was clearly shown that the expected protein bands were more visible and well expressed in the soluble fraction in C. glutamicum than in S. cerevisiae and L. lactis.

Based on the comparative analysis of the GRAS host strains with E. coli, the highly expressed β-glucosidase enzyme of C. glutamicum was selected for biotransformation of the ginsenoside Rg₃-Mix.

TABLE 3

| | | | | | | | | | Relative |
|---|---|---|---|---|---|---|---|---|---|
| | | | | | M. wt of fusion tag | Name of | | Specific | express |
| | | | | Fusion | protein/recombinant | recombinant | Enzymes | activity | enzymes |
| Hosts | Media | Vectors | Inducers | tags | protein (kDa) | enzyme | activity | (U/mg) ⁺ | activities |
| E. coli | LBA | PGEX 4T-1 | IPTG | GST | 26/46 | BglPm | 0.2619 | 10.22 ± 0.62 | 100 |
| C. | LBK | pCES208 | Glucose | His tag | 1/46 | BglPm_C | 0.1976 | 12.54 ± 0.51 | 75.4 |
| glutamicum | | | | | | | | | |
| S. cerevisiae | VPD | pYES2.1 | Galactose | His tag | 1/46 | BglPm_S | 0.03 | 12.92 ± 0.13 | 11.5 |
| Lactococcus | M-17 | pNZ8148 | Nisin | — | —/46 | BglPm_L | 0.244 | ND | 9.3 |
| lactis | | | | | | | | | |

### Example 5. Effect of Sonication on Activities of Enzyme from Each Strain

After the exponential growth of the strains in the particular media as described above, cells were harvested by centrifugation, and pellets were washed twice with a solution consisting of 100 mM sodium phosphate buffer and 1% Triton X-100 (pH 7.0); cells were then re-suspended to a concentration of 1 g/10 mL in lysis buffer (100 mM sodium phosphate buffer [pH 7.0]), in order to measure the difference in the effects of sonication on the enzyme activities derived from each strain. The sonication was performed for the cell suspension of the recombinant E. coli and GRAS hosts strains in a 1.5 mL tube for 20 min to 30 min using Branson digital sonifier 450 (400 W, 70% power, USA).

The activity of crude recombinant β-glucosidase obtained by sonication was determined using 5 mM p-nitrophenyl-β-D-glucopyranoside (pNPGlc) as a substrate. Crude enzyme (20 μL) was incubated in 100 μL of 50 mM sodium phosphate buffer (pH 7.0) containing 5 mM pNPGlc at 37° C., then the reaction was stopped by 0.5 M (final concentration) Na₂CO₃ and the release of p-nitrophenol was measured immediately using a microplate reader at 405 nm (Bio-Rad Model 680; Bio-Rad, Hercules, Calif.). One unit of activity was defined as the amount of protein required to generate 1 μmol of p-nitrophenol per minute. Specific activity was expressed as units per milligram of protein. Protein concentrations were determined using the bicinchoninic acid (BCA) protein assay (Pierce, Rockford, Ill.), with bovine serum albumin (Sigma Aldrich, USA) as the standard. All assays were performed in triplicate.

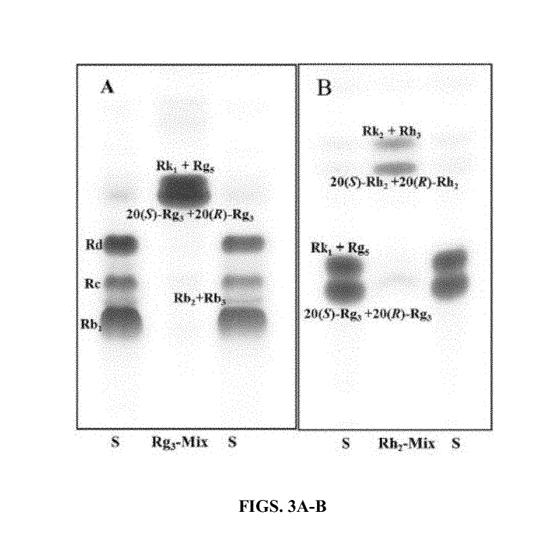

During the investigation of enzymes activities of the GRAS host and recombinant E. coli, which were reacted with 5 mM pNPG, the maximum enzyme activity was obtained by recombinant E. coli after a 10 min period of sonication (further sonication caused loss of enzyme activity) (FIG. 2a). In the case of the GRAS hosts, comparable results were obtained, which showed optimum enzyme activity at 20 min, 25 min, and 15 min for C. glutamicum pCES208 (FIG. 2b), S. cerevisiae pYES2.1 (FIG. 2c), and L. lactis pNZ8148, respectively.

Collectively, these results suggest that the maximum enzyme activity of the GRAS host strains were comparable with recombinant E. coli.

On the basis of the data presented here, it was found that BglPm_C expressed by C. glutamicum had an enzyme activity of 75.4% compared with recombinant BglPm expressed by E. coli (as compared to BglPm_S [11.5%] and BglPm_L [9.3%]), as shown in Table 3. β-Glucosidase (BglPm_C), which was highly expressed by C. glutamicum, was therefore selected for the mass production of edible Rh₂-Mix ginsenosides from Rg₃-Mix.

### Example 6. Biotransformation Activity of Rg₃-Mix Using BglPm_C from C. glutamicum

To verify the bioconversion of Rg₃-Mix by BglPm_C expressed by C. glutamicum harboring pCES208, TLC analysis was carried out at regular intervals. BglPm_C (20 mg/mL) was reacted with an Rg₃-Mix solution at a concentration of 5% (w/v, wet base) in 100 mM sodium phosphate buffer (pH 7.0) at 37° C. The samples were taken at regular time intervals and analyzed via thin layer chromatography (TLC) after pre-treatment.

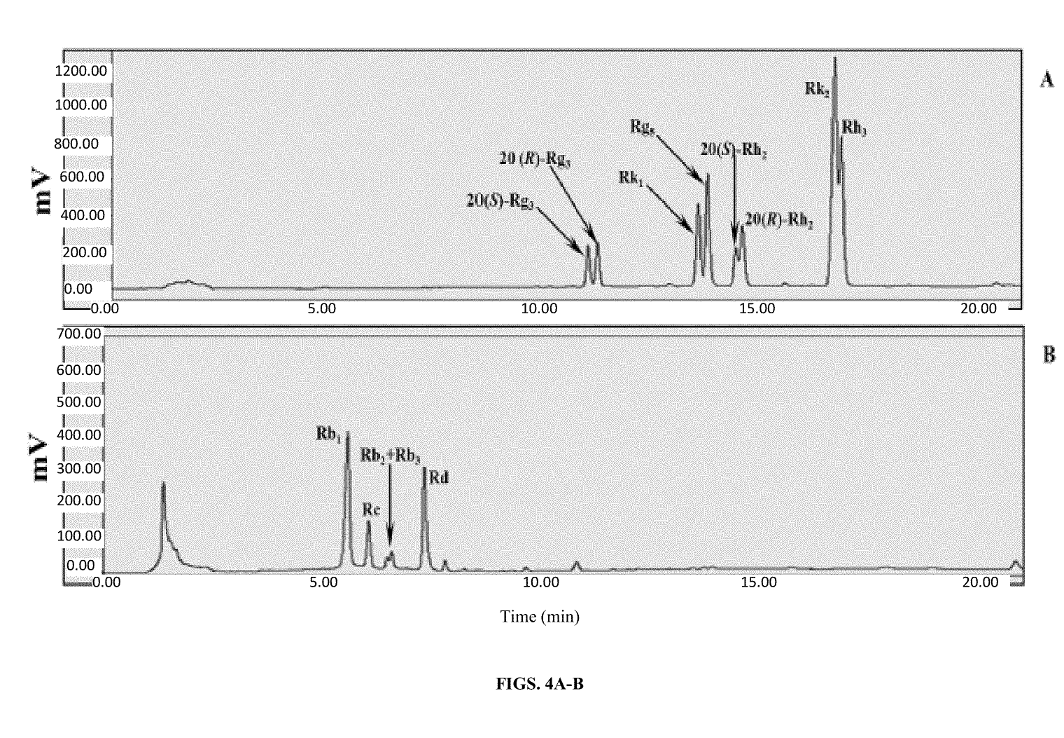

As shown in FIG. 3, the TLC results showed that BglPm_C completely transformed ginsenoside Rg₃-Mix into Rh₂-Mix. The Rf values of ginsenosides Rk₁ and Rg₅ was slightly above the 20(S)-Rg₃ and 20(R)-Rg₃ positions, as shown in FIG. 3a. Rh₂-Mix, which has one glucose moiety removed at the C20 position of Rg₃-Mix, was placed in the upper position of control S (Rg₃-Mix), as shown in FIG. 3b.

### Example 7. Confirmation of Transformation into Ginsenoside Rh₂-Mix Using HPLC Analysis

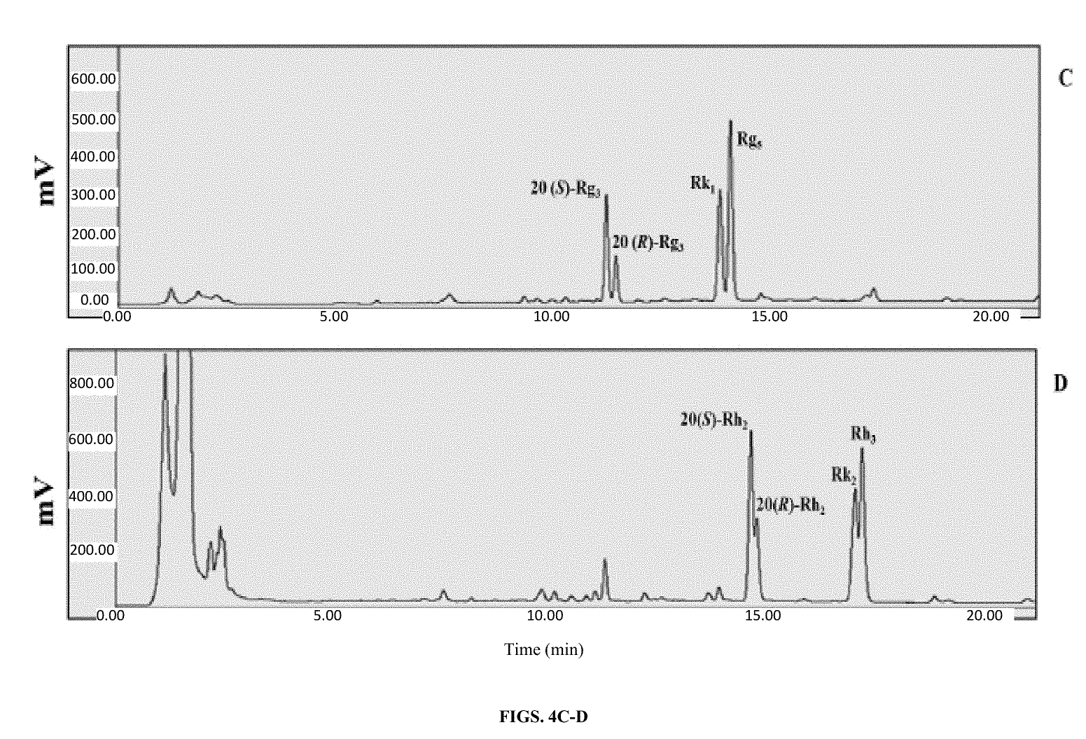

All of the ginsenosides (PPD-Mix, Rg₃-Mix, and Rh₂-Mix) were compared with the ginsenoside standards used in the present invention by HPLC analysis, as shown in FIG. 4a. The ginsenoside PPD-Mix used as the initial substrate is shown in FIG. 4b. For the enzymatic reaction, the PPD-Mix was transformed to the Rg₃-Mix by acid treatment, as shown in FIG. 4c. After 24 hours, the Rh₂-Mix [20(S)—Rh₂, 20(R)—Rh₂, Rk₂, and Rh₃] was produced as a final product from the bioconversion of Rg₃-Mix using the BglPm_C enzyme of C. glutamicum (FIG. 4d).

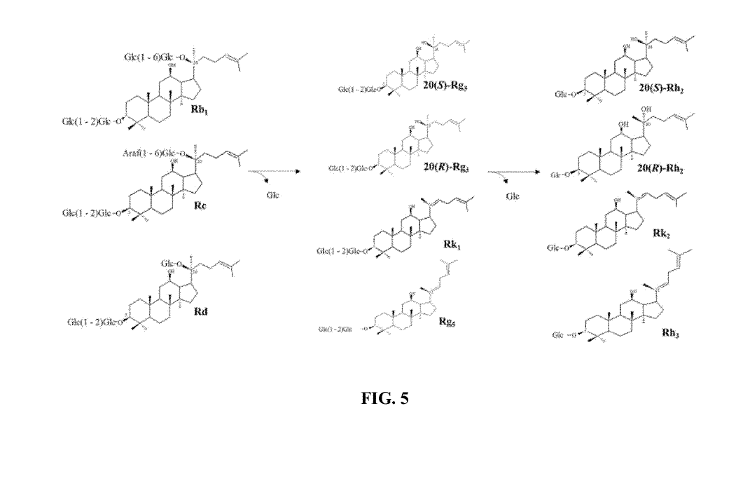

The HPLC analysis revealed that the BglPm_C completely hydrolyzed the Rg₃-Mix within 24 hours. The schematic view of the transformation pathway from PPD-Mix to Rh₂-Mix is shown in FIG. 5.

### Example 8. Scaled-Up Biotransformation of Rg₃-Mix into Rh₂-Mix

### 8-1. Preparation of Recombinant Enzyme (BglPm_C) of C. glutamicum

To obtain high cell density of the recombinant BglPm_C, the LB medium supplemented with kanamycin (50 mg/L final) was used to cultivate the C. glutamicum harboring pCES208 in a 10 L stirred-tank reactor (Biotron GX, Hanil Science Co., Korea) with a 6 L working volume at 400 rpm. Using 100 mM sodium phosphate buffer, the pH value of the medium was adjusted to 7.0. The culture was incubated at 30° C. for 24 hours and the protein expression was induced through the addition of glucose with a final concentration of 10 g/L. After cell density reached an OD of 40 to 42 at 600 nm, the cells were harvested via centrifugation at 8,000 rpm for 20 min. The pellets (50 g) were resuspended in 100 mM sodium phosphate buffer (pH 7.0), and the cells were then broken via sonication (Branson Digital Sonifier, Mexico), and the time was adjusted according to the method described in Example 5. In order to obtain a crude soluble enzyme fraction for the conversion of ginsenosides, unwanted cell debris was removed via centrifugation at 5,000 rpm for 10 min at 4° C. For the enzymatic biotransformation of ginsenoside Rg₃-Mix, the crude recombinant BglPm_C was diluted to the desired concentration with 100 mM sodium phosphate buffer (pH 7.0).

### 8-2. Preparation of BglPm_C and Mass Production of Rh₂-Mix

For the mass production of ginsenoside Rh₂-Mix, the reaction mixture was performed in a 10 L stirred-tank reactor (Biotron GX, Hanil Science Co.) with a 3 L working volume. The reaction mixture was started with a composition of 50 mg/mL (final concentration) of substrate ginsenosides (Rg₃-Mix; total 150 g, wet base) and 20 mg/mL of recombinant BglPm_C in 0.1 M sodium phosphate buffer (pH 6.5 to pH 7.0). The reaction was completed under its optimal conditions of pH 6.5 at 300 rpm for 24 hours. After 24 hours, the ginsenoside Rg₃-Mix [20(S)-Rg₃, 20(R)-Rg₃, Rk₁, and Rg₅] was completely converted to the Rh₂-Mix [20(S)—Rh₂, 20(R)—Rh₂, Rk₂, and Rh₃]. Samples were collected at regular intervals and were analyzed by high performance liquid chromatography (HPLC) in order to determine the time course of the biotransformation of ginsenoside Rg₃-Mix to Rh₂-Mix. In order to remove the unwanted substances, the reaction mixture was centrifuged at 8,000 rpm for 10 min. Most of the ginsenoside Rh₂-Mix precipitated to form a solid, with a small quantity remaining dissolved in the supernatant. 3 L of a 95% ethanol solution was used to dissolve the precipitated ginsenosides Rh₂-Mix thoroughly two times.

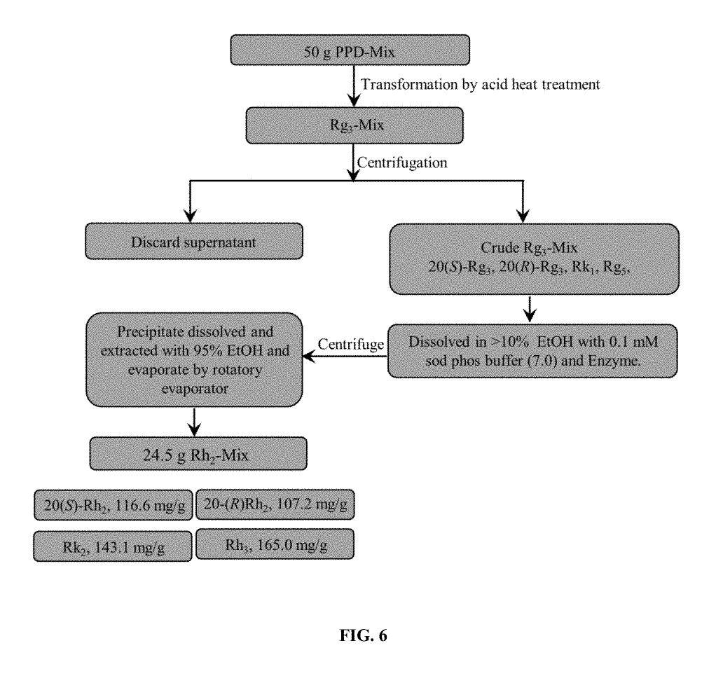

The ginsenoside Rh₂-Mix in the supernatant was evaporated in vacuo in order to create 24.5 g of powdered Rh₂-Mix [20(S)—Rh₂ (116.6 mg/g), 20(R)—Rh₂ (107.2 mg/g) Rk₂ (143.1 mg/g), and Rh₃ (165.0 mg/g)]. Finally, in terms of yield, 24.5 g of Rh₂-Mix was obtained via the conversion of 50 g of PPD-Mix as the initial substrate (FIG. 6).

Based on the results, the method which involves reacting β-glucosidase obtained from recombinant GRAS host strains after the organic acid and heat treatments in PPD-Mix was confirmed to facilitate the mass production of minor ginsenoside Rh₂-Mix.

While the present invention has been described with reference to the particular illustrative embodiments, it will be understood by those skilled in the art to which the present invention pertains that the present invention may be embodied in other specific forms without departing from its spirit or essential characteristics. The described embodiments are to be considered in all respects only as illustrative and not restrictive. The scope of the present invention is therefore indicated by the appended claims rather than by the foregoing description. All changes which come within the meaning and range of equivalency of the claims are to be embraced within the scope of the present invention.

1. A method for mass production of ginsenoside Rh₂-Mix, comprising:
a) treating PPD-Mix with an organic acid to obtain Rg₃-Mix; and
b) treating the Rg₃-Mix obtained in step a) with β-glucosidase.

2. The method of claim 1, wherein the ginsenoside Rh₂-Mix consists of 20(S)—Rh₂, 20(R)—Rh₂, Rk₂, and Rh₃.

3. The method of claim 1, wherein the PPD-Mix consists of Rb₁, Rb₂, Rc, and Rd.

4. The method of claim 1, wherein the Rg₃-Mix consists of 20(S)-Rg₃, 20(R)-Rg₃, Rk₁, and Rg₅.

5. The method of claim 1, wherein step a) further comprises heat treatment.

6. The method of claim 5, wherein the heat treatment is performed at a high temperature of 100° C. to 140° C.

7. The method of claim 1, wherein the β-glucosidase is obtained from a recombinant Generally Recognized As Safe (GRAS) strain.

8. The method of claim 7, wherein the GRAS strain is one selected from the group consisting of Corynebacterium sp., Saccharomyces sp., and Lactococcus sp.

9. The method of claim 8, wherein the Corynebacterium sp. strain is Corynebacterium glutamicum.

10. The method of claim 7, wherein the GRAS strain is a transformant into which a vector comprising a nucleic acid encoding the β-glucosidase is introduced.

11. The method of claim 1, wherein step b) is performed at pH 6.0 to pH 7.0.

12. Rh₂-Mix prepared according to the method of claim 1.

13-14. (canceled)

15. A method for converting PPD-mix into ginsenoside Rh₂-Mix, wherein the method comprises:
a) treating PPD-Mix with an organic acid to obtain Rg₃-Mix; and
b) treating the Rg₃-Mix obtained in step a) with β-glucosidase.

16. The method of claim 15, wherein the Rg₃-Mix consists of 20(S)-Rg₃, 20(R)-Rg₃, Rk₁, and Rg₅.

17. The method of claim 15, wherein the β-glucosidase is obtained from a Generally Recognized As Safe (GRAS) strain.
